(19) United States
(12) Patent Application Publication
(10) Pub. No.: US 2006/0199226 A1
(43) Pub. Date: Sep. 7, 2006

(54) Functional bioluminescence energy resonance transfer (BRET) assay to screen, identify and characterize receptor tyrosine kinase ligands

(76) Inventor: Hans H. Schiffer, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/365,989

(22) Filed: Mar. 1, 2006

Related U.S. Application Data
(60) Provisional application No. 60/658,319, filed on Mar. 2, 2005.

(51) Int. Cl. G01N 33/53; C12Q 1/66; C07H 21/04; C12P 21/06; C12N 9/10; C12N 9/12

(52) U.S. Cl. 435/7.1; 435/8; 435/69.1; 435/193; 435/320.1; 435/325; 536/23.2; 435/194

(57) ABSTRACT

Disclosed herein are methods of evaluating whether a test compound functions as a ligand for a receptor tyrosine kinase. Also provided are methods of making pharmaceutical compositions that include agonists, antagonists, or inverse agonists of receptor tyrosine kinases. Also provides are isolated cells that include a receptor tyrosine kinase and a second protein, wherein the receptor tyrosine kinase contains a fluorescent donor moiety and the second protein contains a fluorescent acceptor moiety.

### RELATED APPLICATIONS

The present application claims priority to the Provisional Application Ser. No. 60/658,319, filed on Mar. 2, 2005, by Hans Schiffer, and entitled “FUNCTIONAL BIOLUMINESCENCE ENERGY RESONANCE TRANSFER (BRET) ASSAY TO SCREEN, IDENTIFY AND CHARACTERIZE, RECEPTOR TYROSINE KINASE LIGANDS,” which is hereby expressly incorporated by reference in its entirety.

### REFERENCE TO SEQUENCE LISTING

The present application is being filed along with duplicate copies of a CD-ROM marked “Copy 1” and “Copy 2” containing a Sequence Listing in electronic format. The duplicate copies of CD-ROM entitled The “Copy 1” and “Copy 2” each contains a file entitled ACADIA.072A.txt created on Mar. 1, 2006 which is 1,146,175 Bytes in size. The information on these duplicate CD-ROMs is incorporated herein by reference in its entirety.

### FIELD OF INVENTION

The present invention relates to a functional bioluminescence resonance energy transfer (BRET) kinase assay for screening and characterizing candidate molecules for their ability to activate or inhibit a kinase.

### BACKGROUND OF THE INVENTION

An important focus of pharmaceutical drug discovery is the identification of surrogate ligands for receptor proteins. Of particular interest in this respect is a subclass of receptor proteins known as receptor tyrosine kinases. The receptor tyrosine kinase (RTK) family is a large group of cell surface receptors that function in the regulation of cell growth, cell differentiation, adhesion, migration and apoptosis (Blume-Jensen and Hunter 2001) (Ullrich and Schlessinger 1990) (Schlessinger 2000) (Hubbard and Till 2000). A number of human diseases have been linked to alterations in RTKs (Akin and Metcalfe 2004) (Verheul and Pinedo 2003) (Corfas et al., 2004). Many RTKs have been identified as oncogenes in transforming retrovirus or human cancers (Hunter 2000) (Shawver et al., 2002) (Muller-Tidow et al., 2004), and recent reports indicate that RTKs may play a critical role in almost all types of human cancer (Shawver et al., 2002) (Prenzel et al., 2000) (Mass 2004). Both naturally occurring and artificial ligands that modulate RTK activity and signaling thus would be of tremendous interest from a therapeutic standpoint with respect to cancer and other diseases. (Haluska and Adjei 2001) (Sawyer et al., 2003). The ability to quickly, efficiently, and effectively screen vast libraries of compounds for particular activities would be greatly advantageous in identifying naturally occurring and/or artificial ligands that modulate RTK activity.

High throughput screening has become a commonly employed strategy to identify novel compounds with particular activities from a diverse chemical library of compounds. Often, high throughput screening assays are either based upon measuring compound binding to defined molecular targets or measuring functional outputs resulting from compound/receptor interactions. However, both binding assays and functional assays have limitations. For example, for various technical reasons, binding assays are preformed in non-physiological conditions. Artificial, non-physiological conditions may impact and influence receptor pharmacology, leading to increased unreliability and difficulty in accurate interpretation of the data. Another drawback arises from the nature of the assay, which measures receptor binding only. Thus, binding competition assays do not provide information regarding the physiological function of ligands, such as whether the ligand functions as an agonist or antagonist. Binding competition assays are also severely constrained by the fact that these assays are only useful in the study of binding sites for which labeled ligands (e.g. radioactive label) are available. Binding competition assays are also not easily amenable to orphan receptors for which ligands have not yet been identified. In addition, purchase, handling and disposal of radioisotopes are major expenses.

Functional assays overcome some of the limitations associated with binding competition assays. For example, functional assays are amenable to screening orphan receptors, and binding sites for which there is no labeled ligand available. Further, since functional assays measure activity output, the data generated enables discrimination between agonist and antagonist ligands (Kenakin 1999).

Although functional assays provide several advantages over binding competition assays, there are still several drawbacks to these assays. Specifically, functional assays are not amenable to the study of receptors for which there is no known cognate signaling pathway, since knowledge of the signal transduction properties of a the receptor, such as an RTK is an important prerequisite to be able to decide which functional assay can be successfully applied. Further, a single RTK may elicit heterogeneous responses, or functional outputs thus rendering measuring activity levels in response to various ligands more difficult.

Several functional assays have been described for receptor tyrosine kinases. Exemplary assays include the quantification of autophosphorylation of RTKs (Olive 2004), measurement of phosphorylation of RTKs and downstream signaling molecules (Olive 2004), measurement of intracellular calcium release (Dupriez et al., 2002), or measurement of RTK dependent cell proliferation (Mosmann 1983) (Bellamy 1992). Functional assays based on each of the above outputs have limitations. Specifically, measuring phosphorylation of RTKs or phosphorylation of downstream signaling proteins, or intracellular calcium changes lacks the throughput needed to perform HTS effectively and requires often expensive supply of antibodies. Furthermore, because the phenomenon of cell proliferation is affected by a large number of gene products, there is an intrinsically high rate of identifying a large percentage of false positives, such as compounds, which are not acting on the RTK of interest. Finally, background activity, or activity due to events other than activity of the receptor tyrosine kinase of interest poses a theoretical limitation in all functional assays.

Newer methods of screening for ligands are based on fluorescence polarization (FP) and time resolved fluorescence (TRF). A drawback of those fluorescence-based assays is the possible auto fluorescence background from the sample studied and the compound tested.

### SUMMARY OF THE INVENTION

Embodiments provide a method of evaluating whether a test compound functions as a ligand for a receptor tyrosine kinase, by providing a cell having a receptor tyrosine kinase and a second protein, wherein the second protein is found within close physical distance to the receptor tyrosine kinase if the test compound is a ligand for the receptor tyrosine kinase; contacting the cell with a test compound; and determining whether the receptor tyrosine kinase and the second protein interact in the presence of the test compound.

Other embodiments provide a method of evaluating whether a test compound functions as a ligand for a receptor tyrosine kinase, by providing an cell having a receptor tyrosine kinase and a second protein, wherein the receptor tyrosine kinase and second protein are within close physical distance to each other; contacting the cell with a test compound, wherein if the test compound is a ligand for the receptor tyrosine kinase, the receptor tyrosine kinase and the second protein will dissociate such that they are no longer within close physical distance to each other; and detecting the interaction between the receptor tyrosine kinase and the second protein.

In aspects of each of the above embodiments, the receptor tyrosine kinase comprises a bioluminescent donor moiety and the second protein comprises a fluorescent acceptor moiety. The receptor tyrosine kinase can be a fusion protein comprising a tyrosine kinase fused to the bioluminescent donor moiety, such as a luciferase, e.g., Renilla luciferase.

In other aspects of each of the above embodiments, the fluorescent acceptor moiety is a GFP moiety, such as GFP2, YFP, CFP, or an isoform or derivative thereof.

In embodiments wherein the RTK comprises a bioluminescent donor moiety, the determination step can comprise calculating the ratio of light emissions from the bioluminescent donor moiety and the fluorescent acceptor moiety.

In aspects of each of the above embodiments, the second protein can be a signaling protein that mediates receptor tyrosine kinase signal transduction. For example, the signaling protein can comprise an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52. Alternatively, the signaling protein can an amino acid sequence that has at least 70% amino acid identity to an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52, wherein the signaling protein retains the ability to interact with a cognate receptor tyrosine kinase. Yet other aspects provide signaling proteins having at least 5 consecutive amino acids of an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52, wherein the signaling protein retains the ability to interact with a cognate receptor tyrosine kinase.

In other aspects of each of the above embodiments the receptor tyrosine kinase can be an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226. In other aspects of each of the above embodiments, the receptor tyrosine kinase can have at least 70% identity to an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, and 226, and wherein the receptor tyrosine kinase retains the ability to mediate ligand binding and signal transduction. Alternatively, in other aspects, the receptor tyrosine kinase comprises an amino acid having at least 5 consecutive amino acids of an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226, and wherein the receptor tyrosine kinase retains the ability to mediate ligand binding and signal transduction.

In aspects of each of the above embodiments, bioluminescence resonance energy transfer (BRET) technology can be utilized.

In aspects of embodiments providing a method of evaluating whether a test compound functions as a ligand for a receptor tyrosine kinase, by providing a cell having a receptor tyrosine kinase and a second protein, wherein the second protein is found within close physical distance to the receptor tyrosine kinase if the test compound is a ligand for the receptor tyrosine kinase; contacting the cell with a test compound; and determining whether the receptor tyrosine kinase and the second protein interact in the presence of the test compound, and embodiments that provide a method of evaluating whether a test compound functions as a ligand for a receptor tyrosine kinase, by providing an cell having a receptor tyrosine kinase and a second protein, wherein the receptor tyrosine kinase and second protein are within close physical distance to each other; contacting the cell with a test compound, wherein if the test compound is a ligand for the receptor tyrosine kinase, the receptor tyrosine kinase and the second protein will dissociate such that they are no longer within close physical distance to each other; and detecting the interaction between the receptor tyrosine kinase and the second protein, the receptor tyrosine kinase can be a fusion protein comprising a tyrosine kinase fused to said bioluminescent donor moiety.

In further aspect, the determination step comprises calculating the ratio of light emissions from the bioluminescent donor moiety and the fluorescent acceptor moiety. In yet other aspects, the second protein is a signaling protein that mediates receptor tyrosine kinase signal transduction. For example, the signaling protein can be an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52. In other aspects, the signaling protein can have an amino acid sequence having at least 70% amino acid identity to an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52, wherein the signaling protein retains the ability to interact with a cognate receptor tyrosine kinase. In other aspects, the signaling protein has at least 5 consecutive amino acids of an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52, wherein the signaling protein retains the ability to interact with a cognate receptor tyrosine kinase.

In other aspects, the receptor tyrosine kinase can be an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226. In yet other aspects, the receptor tyrosine kinase comprises an amino acid sequence having at least 70% identity to an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, and 226, and wherein the receptor tyrosine kinase retains the ability to mediate ligand binding and signal transduction. In other aspects, the receptor tyrosine kinase comprises at least 5 consecutive amino acids of an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226, and wherein said receptor tyrosine kinase retains the ability to mediate ligand binding and signal transduction.

In still other aspects, fluorescent resonance energy transfer (FRET) technology can be utilized.

Other embodiments provide a method of assessing the effect of a test compound on the activity of a receptor tyrosine kinase comprising:
(i) providing a cell expressing one or more receptor tyrosine kinases and one or more second proteins, wherein at least one of the receptor tyrosine kinases or second proteins is expressed from a nucleic acid which has been introduced into the cell;
(ii) contacting the cell with the test compound; and
(iii) determining whether the test compound influences the activity of the receptor tyrosine kinase.

In some aspects, the determining step comprises determining whether the test compound is an agonist, determining whether the test compound is an antagonist, determining whether the test compound is an inverse agonist or determining whether the test compound is a selective modulator.

In other aspects, the test compound is a naturally occurring compound. In yet other aspects, the test compound is a synthetic compound.

Other embodiments provide an isolated cell comprising a receptor tyrosine kinase and a second protein, wherein the receptor tyrosine kinase comprises a bioluminescent donor moiety fusion protein and wherein the second protein comprises a fluorescent acceptor moiety fusion protein, and wherein modulation of the activity of the receptor tyrosine kinase affects the protein protein interactions between the receptor tyrosine kinase and the second protein.

Yet other embodiments provide an isolated cell comprising a receptor tyrosine kinase and a second protein, wherein the receptor tyrosine kinase comprises a fluorescent donor moiety fusion protein and wherein the second protein comprises a fluorescent acceptor moiety fusion protein, wherein the fluorescent donor moiety and the fluorescent acceptor moiety are different, and wherein modulation of the activity of the receptor tyrosine kinase affects the protein protein interactions between the receptor tyrosine kinase and the second protein.

In some aspects, the receptor tyrosine kinase is a fusion protein comprising a receptor tyrosine kinase fused to a fluorescent protein. For example, the fluorescent donor moiety can be a GFP moiety, such as GFP2. In yet other aspects, the fluorescent donor moiety is a YFP moiety, or a CFP moiety or any derivative thereof.

In other aspects, the fluorescent acceptor moiety is a GFP moiety, such as GFP2. In yet other aspects, the fluorescent acceptor moiety is a YFP moiety, or a CFP moiety.

In some aspects, the second protein is a signaling protein that mediates receptor tyrosine kinase signal transduction. In further aspects, the signaling protein comprises an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52. In other aspects, the signaling protein comprises an amino acid sequence having at least 70% amino acid identity to an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52, wherein the signaling protein retains the ability to interact with a cognate receptor tyrosine kinase. In still other aspects, the signaling protein comprises at least 5 consecutive amino acids of an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52, wherein the signaling protein retains the ability to interact with a cognate receptor tyrosine kinase.

In other aspects, the receptor tyrosine kinase comprises an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226. In yet other aspects the receptor tyrosine kinase comprises an amino acid sequence having at least 70% identity to an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226, and wherein the receptor tyrosine kinase retains the ability to mediate ligand binding and signal transduction. In still other aspects the receptor tyrosine kinase comprises at least 5 consecutive amino acids of an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226, and wherein the receptor tyrosine kinase retains the ability to mediate ligand binding and signal transduction.

In other aspects, cell is a eukaryotic cell. In further aspects, the cell is a mammalian cell.

Other embodiments provide method of evaluating whether a test compound functions as a ligand for a receptor tyrosine kinase, by providing an isolated cell comprising a receptor tyrosine kinase fused to a bioluminescent donor moiety and a second protein fused to a fluorescent acceptor moiety, contacting the cell with a substrate, wherein the bioluminescent donor moiety on the receptor tyrosine kinase emits light at a first wavelength in the presence of the substrate, wherein the energy emitted from the bioluminescent donor moiety is transferred to the fluorescent acceptor moiety on the second protein when the fluorescent acceptor moiety is in close proximity to the bioluminescent donor moiety, and wherein the fluorescent acceptor moiety emits light at a second wavelength when the bioluminescent donor moiety transfers energy to the fluorescent acceptor moiety; contacting the cell with a test compound; and measuring the emission of light at the first wavelength and at the second wavelength.

Other embodiments provide a method of making a pharmaceutical composition comprising: performing any one of the methods provided in each of the embodiments described above, identifying a test compound that is an agonist of the receptor tyrosine kinase; and combining the test compound with a pharmaceutically acceptable carrier.

Another embodiment provides a method of making a pharmaceutical composition comprising performing any one of each of the above embodiments providing methods for evaluating whether a test compound functions as a ligand for a receptor tyrosine kinase, or for assessing the effect of a test compound on the activity of a receptor tyrosine kinase, identifying a test compound that is an antagonist of the receptor tyrosine kinase; and combining the test compound with a pharmaceutically acceptable carrier.

Other embodiments provide a method of making a pharmaceutical comprising performing the method of any one of the above embodiments providing methods of evaluating whether a test compound functions as a ligand for a receptor tyrosine kinase, or for assessing the effect of a test compound on the activity of a receptor tyrosine kinase. identifying a test compound that is an inverse agonist of the receptor tyrosine kinase; and combining the test compound with a pharmaceutically acceptable carrier.

Other embodiments provide a method of making a pharmaceutical comprising performing the method of any one of each of the above embodiments that providing methods of evaluating whether a test compound functions as a ligand for a receptor tyrosine kinase, or for assessing the effect of a test compound on the activity of a receptor tyrosine kinase, identifying a test compound that is an selective modulator of the receptor tyrosine kinase; and combining the test compound with a pharmaceutically acceptable carrier

Other embodiments provide a pharmaceutical composition comprising a compound identified by any one the embodiments that provide methods of evaluating whether a test compound functions as a ligand for a receptor tyrosine kinase, or for assessing the effect of a test compound on the activity of a receptor tyrosine kinase, and a physiologically acceptable carrier, diluent, or excipient, or a combination thereof.

### BRIEF DESCRIPTION OF THE FIGURES

FIG. 1A illustrates the primary structure and topology of a typical RTK protein, including the transmembrane domain, kinase domain, and regulatory domain. FIG. 1B, depicts ligand binding induced activation of a receptor tyrosine kinase.

FIG. 2 is an illustration of exemplary RTK signaling pathways: (I) the RAS mitogen-activated protein kinase (RAS/MAP kinase) pathway, (II) the STAT pathway, (III) phosphatidylinositol 3′ kinase-protein B (PI3K-PKB/Akt) pathway and (IV) the phospholipaseC—protein kinase C (PLC-PKC) pathway. Phosphorylated receptor tyrosine kinase molecules recruit and modulate the activity of various signaling molecules, which in turn regulate various cellular functions such as transcription, metabolism, protein synthesis, etc.

FIG. 3 depicts a BRET assay. The host cell is engineered to co-express an RTK-Renilla luciferase fusion protein and an RTK signaling protein-GFP fusion protein (GFP-GRB2).In the absence of ligand, contacting the cells with luciferase substrate (coelenteracine) results in emission of light at 395 nm. Following ligand binding, the RTK-luciferase fusion protein dimerizes and autophosphorylates. In this state, the RTK-luciferase fusion protein recruits the GFP-GRB2 to the RTK-luciferase. The close proximity of Renilla luciferase and GFP2 allows GFP excitation and emission of green light at 510 nm in the presence of substrate.

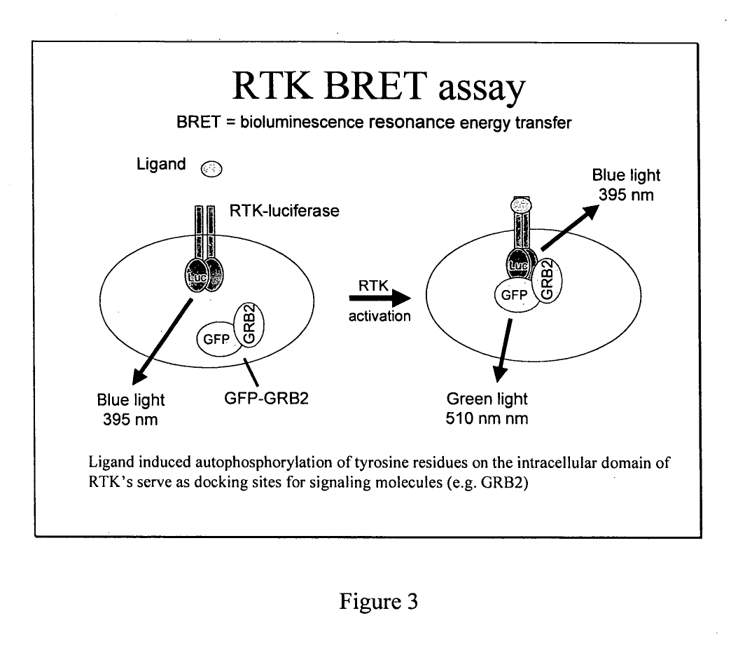

FIG. 4 illustrates a typical BRET assay readout, showing ligand dependent axtivation of the RTK epidermal growth factor receptor (EGFR) using the signaling protein Grb2. The EGFR agonist epidermal growth factor (EGF) leads to a dose-dependent increase in the BRET signal.

FIG. 5 illustrates a typical BRET assay readout, showing ligand dependent inhibition of agonist activated RTK epidermal growth factor receptor (EGFR) using the signaling protein Grb2. Contacting the cells with the EGFR antagonist Iressa (gefitinib) leads to a dose dependent decrease in an agonist evoked BRET signal.

FIG. 6 illustrates the stability of the BRET signal generated in an agonist (EGF) evoked epidermal growth factor receptor (EGFR) over a time period of 24 hours.

FIG. 7 illustrates the stability of the BRET signal generated in an agonist evoked epidermal growth factor receptor (EGFR) after 24 hours using various concentrations of agonist.

FIG. 8 illustrates the stability of the BRET signal generated in an agonist (EGF) evoked epidermal growth factor receptor (EGFR) following treatment with an antagonist (Iressa).

FIG. 9 illustrates the stability of the BRET assay for detection of ligand dependent inhibition of agonist induced activation of receptor tyrosine kinase EGFR. The signaling protein is Grb2.

FIG. 10 illustrates a typical BRET assay readout, showing ligand dependent activation of the RTK epidermal growth factor receptor (EGFR) using the signaling protein Shcp46. The EGFR agonist epidermal growth factor (EGF) leads to a dose-dependent increase in the BRET signal.

FIG. 11 illustrates a typical BRET assay readout, showing ligand dependent inhibition of RTK epidermal growth factor receptor (EGFR) using the signaling protein Schp42. Application of the EGFR antagonist Iressa leads to a dose dependent decrease in an agonist evoked BRET signal. 2 nM epidernal growth factor (EGF) was used as agonist.

FIG. 12 illustrates a typical BRET assay readout, showing ligand dependent activation of RTK epidermal growth factor receptor (EGFR) using the signaling protein STAT5A. The EGFR agonist epidermal growth factor (EGF) leads to a dose-dependent increase in the BRET signal.

### DETAILED DESCRIPTION OF THE PREFERRED EMBODIMENTS

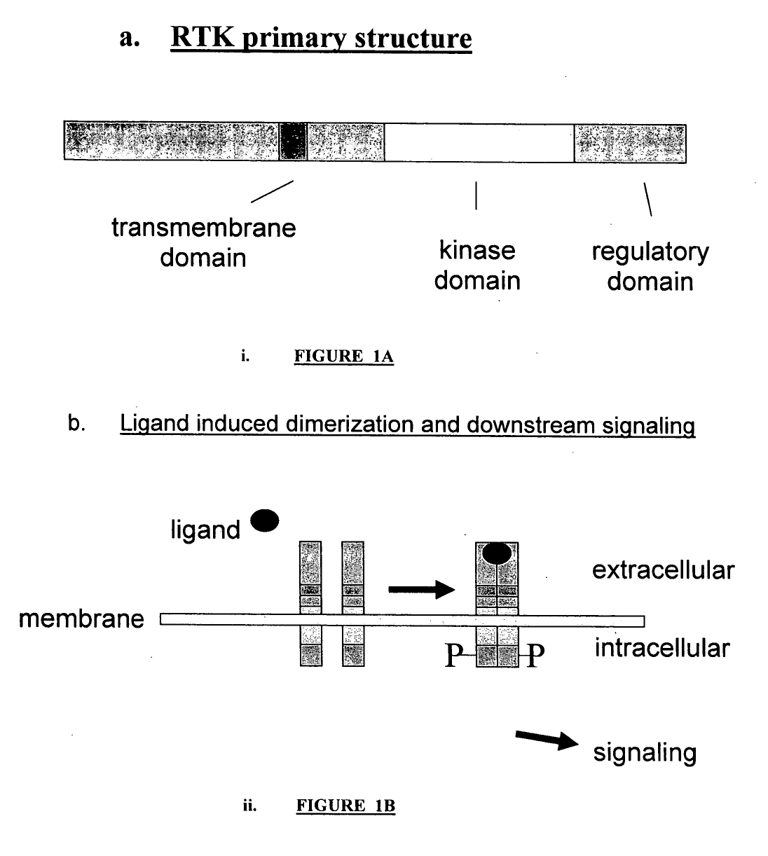

Methods disclosed herein relate to evaluating whether a test compound functions as a ligand for a receptor tyrosine kinase (RTK). Receptor tyrosine kinases (RTKs) are functionally defined as ligand activated kinases that transduce an extracellular signal across the cellular membrane into eukaryotic cells (Blume-Jensen and Hunter 2001) (Ullrich and Schlessinger 1990) (Schlessinger 2000) (Hubbard and Till 2000). A schematic of a typical RTK is depicted in FIG. 1. RTKs have an extracellular domain, a transmembrane domain, a kinase domain and a regulatory domain. Ligand binding occurs via the extracellular domain. The regulatory domain contains tyrosine residues that are phosphorylated through the autocatalytic kinase activity of the kinase domain when the RTK is activated. Numerous receptor kinases have been identified to date in several species, and are amenable to use in the methods disclosed herein. A non-limiting list of some of the receptor tyrosine kinases that have been identified in humans is found in Table 2.

As used herein, the term “ligand” is intended to include any substance that either inhibits or stimulates the activity of a receptor. Ligands can be naturally occurring (e.g., isolatable from a natural source), or synthesized (e.g., manipulated by man). For example in some embodiments synthetic compounds can be generated using combinatorial chemistry. Ligands can be any type of molecule, including but not limited to small molecules, pharmaceuticals, nucleic acids, and peptides. Mechanistically, different types of ligands modulate the activity of receptor kinases in different ways. For example, binding of agonists to RTKs increases their functional activity. On the other hand, binding of antagonist ligands decreases the RTKs' functional activity. Antagonists themselves can function either by inhibiting the action of an agonist or by its own activity (inverse agonist).

Upon binding of an agonist ligand, the RTK can undergo hetero- or homodimer formation, as shown in FIG. 1. Dimerization of the receptor can activate an intrinsic kinase activity of the receptor, which can lead to autophosphorylation of tyrosine residues within the cytoplasmic tail of the receptors (Heldin 1995) (Ullrich and Schlessinger 1990) (McDonald et al., 1995) (Cunningham and Greene 1998). The phosphorylated tyrosine sites can serve as docking sites for a variety of signaling molecules, whose recruitment leads to the activation of several intracellular pathways (Pawson et al., 1993; Pawson and Schlessingert 1993) (Pawson 1995; Pawson 2002). The pattern of tyrosine autophosphorylation determines which pathway is activated, and this pattern is in turn influenced by the identity of the ligands, as well as the identity of the RTK, and whether it is a hetero or homodimer.

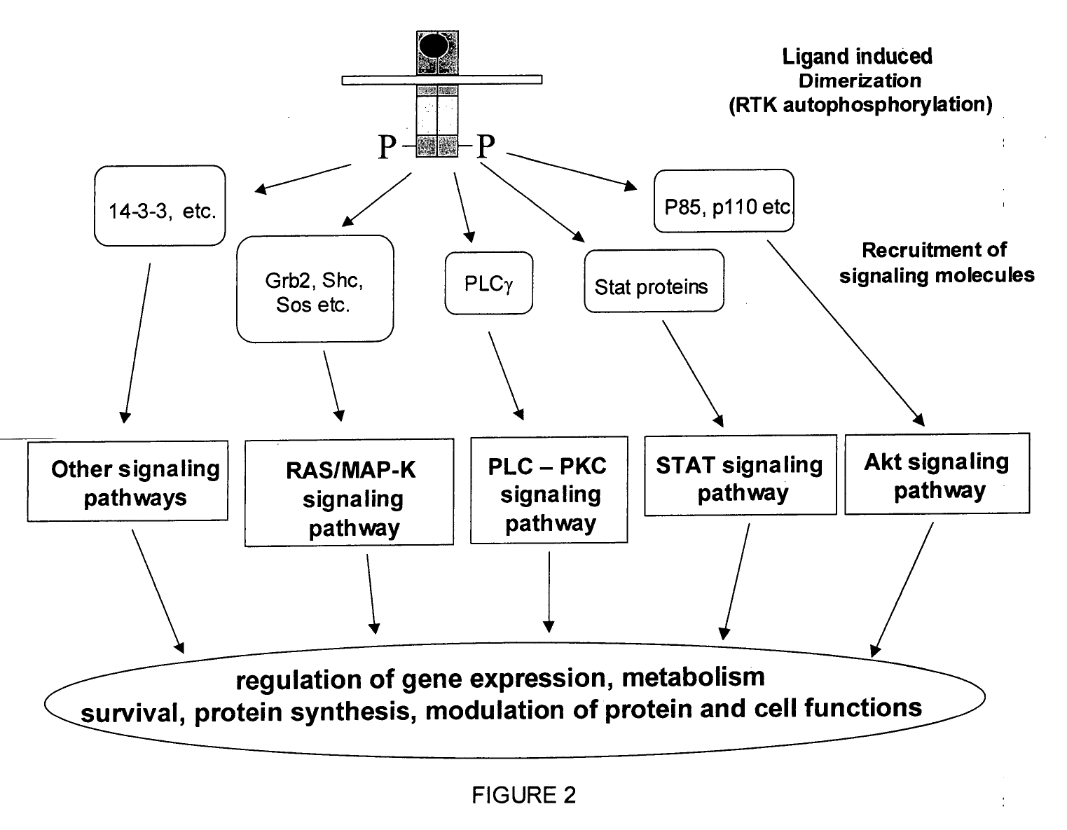

recruitment of signaling molecules to activated RTKs reflects an initial step in the signal transduction pathway (Pawson et al., 1993; Pawson and Schlessingert 1993) (Pawson 1995; Pawson 2002). Proteins involved in RTK biology and signaling may interact with more than one RTK. Several signaling proteins are known to have general functions in RTK biology and interact with many, if not all known RTKs (e.g. Grb2). Other signaling molecules, such as the Ptp-1B, tensinl, Rackl, N-Rage, PSD-95 etc. have a more limited range of cognate RTKs. Each RTK signaling pathway is characterized by a specific set of signaling proteins, which are involved in transducing the ligand induced RTK signal from the receptor downstream into the cell. FIG. 2 shows exemplary sets of signaling proteins interacting with a receptor tyrosine kinase. The particular signaling pathway is dependent upon what signaling molecules interact with the activated receptor tyrosine kinase. As will become clear from the discussion below, the embodiments disclosed herein provide a means of evaluating the effects of test compounds on particular RTK signaling pathways, as well as for identifying “selective modulators”, or compounds that modulate the activity of a particular combination of receptor and one or more signaling molecules, that have mixed agonist/antagonist characteristics. A non-limiting list of examples of RTK signaling molecules is presented in Table 1.

In some embodiments, a suitable host cell is engineered to co-expresses an RTK and a second protein. Any RTK, such as those listed in Table 1, can be expressed in cells in embodiments disclosed herein. Those of skill in the art will understand that variants of RTKs that retain their ligand binding and activation properties, including autokinase and interaction with signaling molecules can also be used in the described embodiments. For example, polypeptides that have at least 97%, at least 95%, at least 90%, at least 85%, at least 80%, or at least 70% sequence identity to, any one of the polypeptides of SEQ ID NO's: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226, that still retain ligand binding and activation properties of those polypeptides can be used in the described embodiments. Alternatively truncated versions of second proteins that have at least 5, 10, 15, 20, 25, 30, 40, 50, 75, 100, or 150, or more than 150 consecutive amino acids of any one of the polypeptides of SEQ ID NO's: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226, that still retain ligand binding and activation properties of those polypeptides can be used in the described embodiments. Identity can be determined using the FASTA version 3.0t78 algorithm with the default parameters. Alternatively, protein identity or similarity can be identified using BLASTP with the default parameters, BLASTX with the default parameters, TBLASTN with the default parameters, or tBLASTX with the default parameters. (Altschul, S.F. et al. Gapped BLAST and PSI-BLAST: A New Generation of Protein Database Search Programs, Nucleic Acid Res. 25: 3389-3402 (1997), the disclosure of which is incroproated herein by reference in its entirety).

The expressed RTK can be in the form of a fusion protein, for example a fusion to a bioluminescent donor moiety, or a fluorescent donor moiety. As used herein, the term bioluminescent donor moiety (BDM) refers to any moiety capable of acting on a suitable substrate to transform chemical energy into light energy. There are a number of different bioluminescent donor proteins that can be employed in this invention. One well characterized example is the class of proteins known as luciferases. Luciferaces proteins catalyze an energy-yielding chemical reaction in which a specific biochemical substance, a luciferin (a naturally occurring substrate), is oxidized by an enzyme having a luciferase activity. Both prokaryotic and eukaryotic organisms including species of bacteria, algae, fungi, insects, fish and other marine forms can emit light energy in this manner and each has specific luciferase activities and luciferins, which are chemically distinct from those of other organisms. Luciferin/luciferase systems are very diverse in form, chemistry and function. For example, there are luciferase activities which facilitate continuous chemiluminescence, as exhibited by some bacteria and mushrooms, and those which are adapted to facilitate sporadic, or stimuli induced, emissions, as in the case of dinoflagellate algae. As a phenomenon, which entails the transformation of chemical energy into light energy, bioluminescence is not restricted to living organisms, nor does it require the presence of living organisms. It is simply a type of chemiluminescent reaction that requires a luciferase activity, which at one stage or another had its origins from a biological catalyst. Hence the preservation or construction of the essential activities and chemicals suffices to have the means to give rise to bioluminescent phenomena. Bioluminescent proteins with luciferase activity are thus available from a variety of sources or by a variety of means. Examples of bioluminescent proteins with luciferase activity may be found in U.S. Pat. Nos. 5,229,285, 5,219,737, 5,843,746, 5,196,524, 5,670,356.

Alternative BDMs include enzymes, which can act on suitable substrates to generate a luminescent signal. Specific examples of such enzymes are β-galactosidase, alkaline phosphatase, β-glucuronidase and β-glucosidase. Synthetic luminescent substrates for these enzymes are well known in the art and are commercially available from companies, such as Tropix Inc. (Bedford, Mass., USA). BDMs can also be isolated or engineered from insects (U.S. Pat. No. 5,670,356).

Depending on the substrate, BDMs emit light at different wavelengths. Non-limiting examples of substrates for BDMs include coelenterazine, benzothiazole, luciferin, enol formate, terpene, and aldehyde, and the like. The BDM moiety can be fused to either the amino terminal or carboxyl terminal portion of the RTK protein. Preferably, the positioning of the BDM domain within the RTK-BDM fusion does not alter the activity of the native protein. RTK-BDM fusion proteins can be tested to ensure that it retains biochemical properties, such as ligand binding and ability to interact with downstream signaling molecules of the native protein. By way of example, a carboxy terminal fusion of the Renilla luciferase gene to the EGFR RTK can be used in the embodiments described herein.

As used herein, the term fluorescent donor moiety (FDM) refers to any moiety that can fluoresce when excited with an appropriate electromagnetic radiation. Non-limiting examples of FDMs include Green Fluorescent Protein (GFP), or isoforms and derivatives thereof such as YFP, EGFP, EYFP and the like (R. Y. Tsien, (1998) Ann. Rev. Biochem. 63:509-544; U.S. Patent Application No. 20050026234). Red fluorescent protein (RFP) such as a Discosoma RFP; or a fluorescent protein related to an RFP, such as a cyan fluorescent protein (CFP), an enhanced CFP, citrine. Preferably, the positioning of the FDM domain within the FDM-second protein fusion does not alter the activity of the native protein.

The second protein can interact directly or indirectly with the RTK. This interaction can be directly dependent upon the activation state of the RTK. For example, the second protein can be an RTK signaling protein. For example, the signaling protein can have the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, or 52, or can have an amino acid sequence that is at least 97%, at least 95%, at least 90%, at least 85%, at least 80%, or at least 70% identical thereto. Alternatively, the signaling molecule can be a truncated version of comprising at least 5, 10, 15, 20, 25, 30, 40, 50, 75, 100, 150, or more than 150 consecutive amino acids of any one of the amino acid sequences of SEQ ID NO: 2, 4, 6, 8, 10, 12, 14, 16, 18, 20, 22, 24, 26, 28, 30, 32, 34, 36, 38, 40, 42, 44, 46, 48, 50, or 52 that retains RTK interaction properties of the native form of the protein. In yet other aspects, the second protein can be the receptor binding domain of any of the polypeptides of SEQ ID NO's: 2, 4, 6, 8, 10, 12, 14, 16, 18, 20, 22, 24, 26, 28, 30, 32, 34, 36, 38, 40, 42, 44, 46, 48, 50, or 52. Identity can be determined using the FASTA version 3.0t78 algorithm with the default parameters. Alternatively, protein identity or similarity can be identified using BLASTP with the default parameters, BLASTX with the default parameters, TBLASTN with the default parameters, or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incroproated herein by reference in its entirety).

The expressed second protein can be a fusion protein, for example a fusion to a fluorescent acceptor moiety (FAM). The term “acceptor” refers to a quencher that operates via energy transfer. Acceptors may re-emit the transferred energy as fluorescence and are “fluorescent acceptor moieties”. Examples of FAMs, include Green Fluorescent Protein (GFP), or isoforms and derivatives thereof such as YFP, EGFP, EYFP and the like (R. Y. Tsien, (1998) Ann. Rev. Biochem. 63:509-544). Preferably, the positioning of the FAM domain within the FAM-second protein fusion does not alter the activity of the native protein. FAM-second protein fusion proteins can be tested to ensure that it retains biochemical properties of the cognate native protein, such as interaction with RTKs. By way of example, an amino terminal fusion of the GFP protein to Gbr2, Shpc42 or STAT5 can be used in the embodiments described herein.

In aspects of the invention wherein the RTK comprises an FDM, the second protein preferably does not comprise an FAM that is identical to the FDM of the RTK.

In some embodiments, the RTK can comprise a FAM, and the second protein can comprise an BDM or FDM. In aspects of the invention wherein the RTK comprises an FAM, the second protein preferably does not comprise an FDM that is identical to the FAM of the second protein.

In general, DNA sequences encoding one or more receptor tyrosine kinases and DNA sequences encoding second proteins, such as signaling proteins can be cloned into suitable expression vectors, such as plasmids, viral particles and phage, that are compatible with the host cells used in the described embodiments. Those of skill in the art will understand that any of a wide variety of expression systems can be used in the described embodiments, and that expression vectors carry elements necessary for the expression of the proteins in the appropriate host cells, such as promoter sequences, ribosome binding sites. The vector can also comprise elements such as polyadenylation signals, transcriptional enhancer sequences, translational enhancer sequences, origin of replication and integration sequences.

Expression vectors can be engineered using well-known techniques in recombinant DNA technology to create in-frame fusions of the protein of interest (i.e., a receptor tyrosine kinase or a signaling protein) to BDMs or FAMs. Vectors and recombinant DNA techniques are well known to those of skill in the art (cf., for instance Sambrook et al., Molecular Cloning: A laboratory Manual, Cold Spring Harbor Laboratory, Cold Spring Harbor, N.Y., 1989). The RTK and second protein can be expressed from the same vector, or can be expressed from different vectors.

Methods well known to those of skill in the art can be used to introduce the expression vectors into suitable host cells. Methods of transfecting mammalian cells and expressing DNA sequences introduced in the cells are described in e.g., Kaufman and sharp, J. Mol. Biol. 159 (1982), 601-621; Southern and Berg, J. Mol. Appl. Genet. 1 (1982), 327-341; Loyter et al., Proc. Natl. Acad. Sci USA 79 (1982), 422-426; Wiggler et al., Cell 14 (1978), 725; Corsaro and Pearson, Somatic Cell Genetics 7 (1981), 603, Graham and ver der Eb, Virology 52 (1973), 456; Neumann et al., EMBO J. 1 (1982), 841-845; and Wigler et al., Cell 11, 1977, pp. 223-232. Those skilled in the art will understand that expression vectors can be transiently introduced into host cells or can be stably maintained within host cells. Suitable host cells include but are not limited to eukaryotic cells such as yeast cells, insect cells, or mammalian cells such as CHO, VERO, BHK, HeLa, COS, MDCK, HEK293, NIH-3T3, WI38 cells and the like.

Cells expressing RTK proteins and second proteins can be contacted with test compounds. Test compounds can be used whether they are used to test known compounds or to identify new compounds that interact with the receptor. For example, test compounds can be used which include, but are not limited to, small molecules, pharmaceuticals, nucleic acids, peptides, antibodies, ligands, agonists, and antagonists. Following exposure to the test compound, the interaction between the RTK and the second protein can be assessed. As described above, the extent of interactions between the RTK and the second protein is directly correlated to the activation state (i.e., tyrosine phosphorylation pattern) of the RTK.

In some embodiments, the interaction between the RTK and second protein is functionally linked to a measurable output based on a bioluminescence resonance energy transfer (BRET). (Xu et al., 1999) (Bertrand et al., 2002) (Xu et al., 1999) (Vrecl et al., 2004) (Heding 2004). BRET based assays can be used to monitor the interaction of proteins having a bioluminescent donor moiety with proteins having a fluorescent acceptor moiety, such as in the disclosed embodiments. Briefly, cells expressing a RTK-BDM fusion will convert the substrate's chemical energy into light. If there is FAM in close proximity to the RTK-BDM fusion, then the cells will emit light at a certain wavelength. For example, BRET based assays can be used to assess the interaction between a RTK-luciferase fusion and a GFP2 fusion protein to a signaling protein. Treating cells expressing an RTK Renilla luciferase (Rluc) fusion with the substrate coelenteracine 400A (Hart et al., 1979) results in emission of light at 395 nm. Activation of the RTK, for example by agonist binding, results in interaction between the RTK and second protein. The BDM can transfer energy to the FAM if donor and acceptor are close enough in physical distance (<100 angstrom) (Ward and Cormier 1979). With the FAM GFP2 in close proximity to the BDM Rluc, energy is transferred from the luciferase to GFP2, thereby exciting the GFP2 moiety and causing GFP2 to emit of green light at 510 nm. The efficiency of the energy transfer between Rluc and GFP2 can be measured by determine the ratio between the light emission of the fluorescence acceptor moiety and the bioluminescence donor. The BRET signal is expressed as the ratio between the GFP2 and luciferase light emissions. Ligands or compounds that modulate the RTK activity will cause changes in this BRET ratio due to different amounts of RTK-Rluc/GFP2-second protein complexes in the cell.

In other embodiments, the interaction between the RTK and second protein is functionally linked to a measurable output based on a fluorescence resonance energy transfer (FRET). As with BRET, FRET assays involve exploiting the ability to detect the proximity of two fluorophores to each other. FRET can be performed as described in Miyawaki and Tsien, 2000, Methods Enzymol. 327: 472-500, which is herein incorporated by reference in its entirety. For example, FRET can be used to measure the interaction between an RTK comprising an FDM and a second protein comprising an FAM. Ligands or compounds that modulate the RTK activity will cause changes in the FRET ratio due to different amounts of RTK-Rluc/GFP2-second protein complexes in the cell.

Any of the above methods can be used to measure association or dissociation of the RTK and second protein. For example, BRET and FRET measurements of cells contacted with test compounds can be compared to the BRET and FRET measurements of cells that have not been contacted with a test compound.

Any of the methods disclosed above can be used to identify compound that affect the ability of known agonists, antagonists, inverse agonists, and selective modulators, to effect the activity of RTKs. For example, any of the above methods can be performed on cells in the presence of known agonists or antagonists.

In another aspect, the present invention relates to a pharmaceutical composition comprising a compound that has been identified by any of the methods disclosed above, as described above, and a physiologically acceptable carrier, diluent, or excipient, or a combination thereof.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporaton of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into the are of pain, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, or between about 25% and 250%, or between 50% and 100% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

It will be understood by those of skill in the art that numerous and various modifications can be made without departing from the spirit of the present invention. Therefore, it should be clearly understood that the forms of the present invention are illustrative only and are not intended to limit the scope of the present invention.

TABLE I

| | | | | NCBI |
|---|---|---|---|---|
| | Gene | | Protein | Accession |
| Gene | SEQ ID NO. | Protein | SEQ ID NO. | Number |
| GRB2 | 1 | grb2 | 2 | NM_002086 |
| GRB4 | 3 | grb4 | 4 | NM_003581 |
| GRB7 | 5 | grb7 | 6 | D43772 |
| GRB10 | 7 | grb10 | 8 | NM_005311 |
| GRB14 | 9 | grb14 | 10 | L76687 |
| SHC1 | 11 | Shc1 | 12 | NM_003029 |
| SHC2 | 13 | Shc2 | 14 | XM_375550 |
| SHC3 | 15 | Shc3 | 16 | NM_016848 |
| STAT1 | 17 | Stat1 | 18 | NM_007315 |
| STAT2 | 19 | Stat2 | 20 | NM_005419 |
| STAT3 | 21 | Stat3 | 22 | NM_139276 |
| STAT4 | 23 | Stat4 | 24 | NM_003151 |
| STAT5A | 25 | Stat5A | 26 | NM_003152 |
| STAT5B | 27 | Stat5B | 28 | NM_012448 |
| STAT6 | 29 | Stat6 | 30 | NM_003153 |
| YWHAZ | 31 | 14-3-3zeta | 32 | NM_003406 |
| PIK3R1 | 33 | p85alpha | 34 | NM_181523 |
| PIK3R2 | 35 | p85beta | 36 | NM_005027 |
| CBL | 37 | c-Cbl | 38 | NM_005188 |
| CBLB | 39 | Cblb | 40 | NM_170662 |
| CBLC | 41 | Cblc | 42 | NM_012116 |
| RGS4 | 43 | Rgs4 | 44 | NM_005613 |
| PLCG1 | 45 | PlCgamma1 | 46 | NM_002660 |
| PTPN1 | 47 | Ptp-1B | 48 | NM_002827 |
| SH3KBP1 | 49 | Cin85 | 50 | NM_031892 |
| TENC1 | 51 | tensin1 | 52 | NM_015319 |
| RICS | 53 | Grit | 54 | NM_014715 |
| GNB2L1 | 55 | Rack1 | 56 | NM_006098 |
| RGS19IP1 | 57 | Gipc | 58 | NM_005716 |
| AHSG | 59 | Alpha2-HSG | 60 | NM_001622 |
| MAGED1 | 61 | NRAGE | 62 | NM_001005332 |
| ABL1 | 63 | c-abl | 64 | NM_005157 |
| ERBB2IP | 65 | Erbin | 66 | NM_001006600 |
| DLG4 | 67 | PSD95 | 68 | NM_001365 |
| ENPP1 | 69 | PC-1 | 70 | NM_006208 |
| FLJ14768 | 71 | Fiz1 | 72 | NM_032836 |
| RPL13A | 73 | p23 | 74 | NM_012423 |
| CAV1 | 75 | caveolin | 76 | NM_001753 |
| CSK | 77 | c-src | 78 | NM_004383 |
| IRS1 | 79 | IRS-1 | 80 | NM_005544 |
| RASA1 | 81 | Gap | 82 | NM_002890 |
| VAV1 | 83 | Vav1 | 84 | NM_005428 |
| VAV2 | 85 | Vav2 | 86 | NM_003371 |
| VAV3 | 87 | Vav3 | 88 | NM_006133 |
| CRK | 89 | Crk | 90 | NM_005206 |
| NCK1 | 91 | Nck1 | 92 | NM_006153 |
| NCK2 | 93 | Nck2 | 94 | NM_003581 |
| PTPN6 | 95 | Shp1 | 96 | NM_002831 |
| LCP2 | 97 | Slp76 | 98 | NM_005565 |
| SH2D1A | 99 | Sap | 100 | NM_002351 |
| SOCS1 | 101 | Socs1 | 102 | NM_003745 |
| SOCS2 | 103 | Socs2 | 104 | NM_003877 |
| SOCS3 | 105 | Socs3 | 106 | NM_003955 |
| SOCS4 | 107 | Socs4 | 108 | NM_080867 |
| SHCP46 | 229 | Shcp46 | 230 | |

TABLE 2
Human RTK family

| | | | NCBI | Gene | Protein |
|---|---|---|---|---|---|
| Sub- | | | Accession | SEQ ID | SEQ ID |
| family | Gene | Synonyms | Number | NO. | NO. |
| EGFR | EGFR | HER | NM_005228 | 109 | 110 |
| | ERBB2 | HER2 | M11730.1 | 111 | 112 |
| | ERBB3 | HER3 | NM_001982.1 | 113 | 114 |
| | ERBB4 | HER4 | NM_005235.1 | 115 | 116 |
| INSR | IGF1R | JTK13 | NM_000875 | 117 | 118 |
| | INSR | IR | NM_000208.1 | 119 | 120 |
| | INSRR | IRR | NM_014215 | 121 | 122 |
| PDGFR | FLT3 | FLK2 | NM_004119 | 123 | 124 |
| | CSF1R | FMS | NM_005211 | 125 | 126 |
| | KIT | CKIT | NM_000222 | 127 | 128 |
| | PDGFRA | | NM_006206 | 129 | 130 |
| | PDGFRB | JTK12 | J03278 | 131 | 132 |
| FGFR | FGFR1 | FLT2 | NM_023105 | 133 | 134 |
| | FGFR2 | KGFR | NM_023030 | 135 | 136 |
| | FGFR3 | HBGFR | NM_000142 | 137 | 138 |
| | FGFR4 | | NM_002011 | 139 | 140 |
| VEGFR | VEGFR1 | FLT1 | NM_002019 | 141 | 142 |
| | VEGFR2 | FLK1 | NM_002253 | 143 | 144 |
| | VEGFR3 | FLT4 | NM_182925 | 145 | 146 |
| EPH | EPHA1 | EPH | NM_005232.2 | 147 | 148 |
| | EPHA2 | ECK | NM_004431.2 | 149 | 150 |
| | EPHA3 | HEK | NM_005233.3 | 151 | 152 |
| | EPHA4 | SEK1 | NM_004438.3 | 153 | 154 |
| | EPHA5 | EHK1 | L36644 | 155 | 156 |
| | EPHA6 | EHK2 | AL133666 | 157 | 158 |
| | EPHA7 | MDK1 | NM_004440.2 | 159 | 160 |
| | EPHA8 | EEK | NM_020526.3 | 161 | 162 |
| | EPHB1 | ELK | NM_004441.2 | 163 | 164 |
| | EPHB2 | NUK | NM_004442.4 | 165 | 166 |
| | EPHB3 | HEK2 | NM_004443 | 167 | 168 |
| | EPHB4 | HTK | NM_004444 | 169 | 170 |
| | EPHB5 | CEK9 | not yet | | |
| | | | found in | | |
| | | | human | | |
| | EPHB6 | MEP | NM_004445 | 171 | 172 |
| TRK | NTRK1 | TRKA | M23102 | 173 | 174 |
| | NTRK2 | TRKB | AF400441.1 | 175 | 176 |
| | NTRK3 | TRKC | U05012.1 | 177 | 178 |
| | p75 | NGFR | NM_002507.1 | 179 | 180 |
| AXL | AXL | UFO | NM_021913 | 181 | 182 |
| | MER | NYK | NM_006343 | 183 | 184 |
| | TYR03 | SKY | NM_006293 | 185 | 186 |
| TIE | TIE1 | TIE | NM_005424 | 187 | 188 |
| | TIE2 | TEK | NM_000459 | 189 | 190 |
| MET | MET | HGFR | J02958 | 191 | 192 |
| | RON | MST1R | NM_002447 | 193 | 194 |
| DDR | DDR1 | CAK | NM_013993 | 195 | 196 |
| | DDR2 | TKT | NM_006182 | 197 | 198 |
| RET | RET | MEN2A/B | X12949 | 199 | 200 |
| ROS | ROS1 | MCF3 | NM_002944 | 201 | 202 |
| ALK | ALK | Ki1 | NM_004304 | 203 | 204 |
| | LTK | TYK1 | NM_002344 | 205 | 206 |
| ROR | ROR1 | NTRKR1 | NM_005012 | 207 | 208 |
| | ROR2 | NTRKR2 | NM_004560 | 209 | 210 |
| RYK | RYK | | S59184 | 211 | 212 |
| PTK7 | PTK7 | CCK4 | NM_002821 | 213 | 214 |
| MUSK | MUSK | | NM_005592 | 215 | 216 |

### EXAMPLES

The invention described and claimed herein is not to be limited in scope by the specific embodiments herein disclosed, since these embodiments are intended as illustrations of several aspects of the invention. Any equivalent embodiments are intended within the scope of this invention. Indeed, various modifications of the embodiments in addition to those shown and described herein will become apparent to those skilled in the art from the foregoing description. The appended claims are intended to cover such modifications.

The present invention is further described in the following Examples, that are not in any way intended to limit the scope of the invention as claimed.

### Example 1

### General Protocol for Using BRET to Detect Agonist and Antagonist Activities on Receptor Tyrosine Kinases

In vivo assays in cells expressing BDM tagged receptor RTK proteins and FAM tagged signaling proteins were developed to detect the effects of various types of compounds on RTK activity.

Recombinant DNA techniques are used to isolate a nucleic acid encoding an RTK from any source, for example, any of the RTK's listed in Table 2. Standard recombinant DNA protocols are used to clone the nucleic acids encoding the RTK into any plasmid that facilitates the expression of an in frame fusion of the RTK to a bioluminescent donor moiety.

Recombinant DNA techniques are used to isolate a nucleic acid encoding a signaling protein from any source, for example, any of the signaling proteins listed in Table 1. Standard recombinant DNA protocols are used to clone the nucleic acid encoding the signaling molecule into any plasmid that facilitates the expression of an in frame fusion of the signaling protein to a fluorescent acceptor moiety.

Any means now known or discovered in the future are used to co-introduce the expression constructs into a host cell. The host cells are propagated using known culturing methods, and both fusion proteins are co-expressed in the recombinant host cells.

To detect agonist activity, samples of the transfected cells are added to a 96-well plate (Costar 3912). Various concentrations of known agonists or test compounds are added to the wells with the cell samples. Following a short incubation period, for example about 20 minutes, the BRET reaction is started by adding substrate (for example coelenteracine), to the cell sample. The emission of light at various wavelengths is measured on a Mithras LB940 plate reader (Berthold, Germany) or instrument with similar capabilities. For example, BRET optimized filters (Berthold, Germany) are used to detect luciferase emission at 395 nm and flourescent emission at 510 nm wavelength. The emissions are measured for one second each. Data from the BRT BRET assays are fit to the equation R=D+(A−D)/(1+(x/c)), where A+minimum response, D=maximum response and c=EC50 (R=response, x=concentration of ligand) using the Prism 4.0 software (GraphPad Software, Dan Diego, Calif., USA). The readings from three wells is used to generate each data point. The data points for cells treated with test compounds is compared to the data points for cells not treated with known agonists or antagonists. A comparison of the BRET signals to untreated cells, or cells treated with known agonists or antagonists, is used to characterize the test compound as a ligand, as well as if the ligand is an agonist, antagonist, or inverse agonist of the RTK.

To detect antagonist activity, samples of the transfected cells are added to a 96 well plate. A known agonist is added to the cells, and the cells are incubated for a period of time, such as for 10 minutes. Following incubation with the known agonist, various concentrations of a known antagonist or test compound are added to the cell samples. Cells are incubated with the known antagonist or test compound, for example for 10 minutes. Following a short incubation period, for example about 20 minutes, the BRET reaction is started by adding substrate (for example coelenteracine), to the cell sample. The emission of light at various wavelengths is measured on a Mithras LB940 plate reader (Berthold, Germany) or instrument with similar capabilities. For example, BRET optimized filters (Berthold, Germany) are used to detect luciferase emission at 395 nm and fluorescent emission at 510 nm wavelength. The emissions are measured for one second each. Data from the BRT BRET assays are fit to the equation R=D+(A−D)/(1+(x/c)), where A+minimum response, D=maximum response and c=EC50 (R=response, x=concentration of ligand) using the Prism 4.0 software (GraphPad Software, Dan Diego, Calif., USA). The readings from three wells are used to generate each data point. The data points for cells treated with test compounds are compared to the data points for cells not treated with known agonists. A comparison of the BRET signals to untreated cells, or cells treated with known agonists, is used to characterize the test compound as a ligand, as well as if the ligand is an antagonist of the RTK.

To detect inverse agonist activity, recombinant DNA techniques are used to isolate a nucleic acid encoding an RTK with constitutive activity (i.e., in an active state in the absence of ligand binding) from any source, for example, any of the RTK's listed in Table 2. Standard recombinant DNA protocols are used to clone nucleic acids encoding the constitutive RTK into any plasmid that facilitates the expression of an in frame fusion of the constitutive RTK to a bioluminescent donor moiety.

Recombinant DNA techniques are used to isolate a nucleic acid encoding a signaling protein from any source, for example, any of the signaling proteins listed in Table 1. Standard recombinant DNA protocols are used to the signaling molecule PCR products into any plasmid that facilitates the expression of an in frame fusion of the signaling protein to a fluorescent acceptor moiety.

The expression constructs encoding the constitutive RTK fusion and the signaling protein fusion are co-introduced into a host cell. The co-transfected host cells are propagated using known culturing methods, and both fusion proteins are co-expressed in the recombinant host cells. Samples of the transfected cells are added to a 96 well plate. Parallel samples are cells are either untreated, or incubated with a test compound. Following a short incubation period, for example about 20 minutes, the BRET reaction is started by adding substrate (for example coelenteracine), to the cell sample. The emission of light at various wavelengths is measured on a Mithras LB940 plate reader (Berthold, Germany) or instrument with similar capabilities. For example, BRET optimized filters (Berthold, Germany) are used to detect luciferase emission at 395 nm and fluorescent emission at 510 nm wavelength. The emissions are measured for one second each. Data from the BRT BRET assays are fit to the equation R=D+(A−D)/(1+(x/c)), where A+minimum response, D=maximum response and c=EC50 (R=response, x=concentration of ligand) using the Prism 4.0 software (GraphPad Software, Dan Diego, Calif., USA). The readings from three wells are used to generate each data point. The data points for cells treated with test compounds are compared to the data points for cells not treated with known agonists. A comparison of the BRET signal of cells treated with test compound to untreated cells is used to characterize the test compound as an inverse agonist of the RTK.

To determine whether a compound functions as a selective modulator of a particular an RTK, two sets of recombinant cells are constructed. Standard recombinant DNA methods described above are used to construct an expression vector encoding an RTK from any source, for example, any of the RTK's listed in Table 2. Nucleic acids encoding the RTK are cloned into any plasmid that facilitates the expression of an in frame fusion of the RTK to a bioluminescent donor moiety. Methods described above are used to construct at least two different expression vectors for the expression of an in frame fusions of signaling proteins from any source, for example, any of the signaling proteins listed in Table 1, to a fluorescent acceptor moiety.

Cells are co-transfected with the RTK fusion and one of the expression constructs using methods described above, to create recombinant cells that express different RTK/signaling protein fusion pairs. Recombinant host cells with different pairs of RTK/signaling fusions are propagated as described above. For each type of recombinant host cell with a particular RTK/signaling protein fusion pair, parallel cell samples are added to a 96 well plate. For each type of recombinant host cell, parallel cell samples are analyzed by incubating some samples with a test compound, and incubating parallel samples under the same conditions without the addition of a test compound. The BRET assay is initiated and performed as described above on all cell samples. For each type of recombinant cell (i.e., that is co-transfected with the RTK and different signaling protein fusions), the effect of the addition of test compound to recombinant cells harboring one type of RTK/signaling molecule fusion pair is measured by comparing the BRET signal in cell samples treated or untreated with test compound. The effect of the test compound on each type of recombinant cell (e.g., each RTK/signaling pair) is compared. The comparison is used to characterize the test compound as a selective modulator, if the test compound has differential effects on the BRET signal for the different type of cells.

### Example 2

### Protocol for Using BRET to Detect Agonist and Antagonist Activities on the EGFR Receptor Tyrosine Kinase

EGFR is a membrane localized receptor tyrosine kinase, which autophosphorylates several tyrosine residues in the intracellular carboxyl-terminus upon activation by the epidermal growth factor (EGF) (Holbro and Hynes 2004) (Mass 2004). These phosphorylated tyrosine residues serve as docking sites for various signaling molecules that are involved in mediating signals downstream into the cell. The epidermal growth factor receptor biding protein 2 (Grb2) is a SH2 and SH3 domain containing RTK signaling protein that binds to phosphorylated tyrosine residues in the carboxyl terminus of EGFR and that links EGFR activity to the RAS/MAP kinase-signaling pathway (Pawson 1995). STAT5A is another signaling protein that interacts with EGFR RTK.

To create an EFGR-luciferase fusion protein (SEQ ID NO: 226), Pfu Turbo polymerase (Stratagene, USA) was used in a polymerase chain reaction to arnplify EGFR cDNA. Total RNA derived from the cancer cell line PC-3 (Ambion, USA) was used as the template DNA. The primers EGFR.LucN3.S (CAGCGGCTAGCGCGATGCGACCCTCCGGG)(SEQ ID NO:227) and EGFR.LucN3.AS (CAGCTGGTACCTGCTCCAATAAATTCACTGCTTTG)(SEQ ID NO:228) were used in the PCR reaction. The EGFR.LucN3.AS primer eliminates the EGFR cDNA stop codon to to facilitate the creation of an in frame fusion to the luciferase protein. The PCR product was cloned directly into the TOPO™ vector pCR2.1 (Invitrogen) to create pTOPO-EGFR. pTOPO-EGFR was digested with NheI and KpnI. The EGFR DNA insert was isolated and ligated into the NheI and KpnI sites of the luciferase plasmid pRLucN3 (Perkin Elmer). The final plasmid contains a nucleic acid encoding an in-frame carboxy-terminal EGFR-Renilla luciferase fusion protein (SEQ ID NO: 225; SEQ ID NO:226). The resulting fusion protein EGFR-luciferase behaves functionally identical to the non-tagged EGFR (data not shown).

To create the GRB2-GFP fusion protein (SEQ ID NO:217), GRB2 cDNA was amplified by PCR using Pfu Turbo polymerase (Stratagene, USA) with the following primers: GRB2.GFP2.S (CAGCGGAATTCGGAATGGAAGCCATCGCCAAATATG)(SEQ ID NO:219) and GRB2.GFP2.AS (CAGCTCCGCGGTTAGACGTTCCGGTTCACGGGG)(SEQ ID NO:220) The GRB2.GFP2.AS primer is engineered to eliminate the GRB2 cDNA stop codon to facilitate the creation of an in frame fusion to the GFP2 protein. The PCR product was subcloned blunt ended into the TOPO™ vector pCR2.1 (Invitrogen, Carlsbad, Calif.), to create pGRB2-TOPO. pGRB2-TOPO was digested with EcoRI and SacII and the Grb2 fragment was isolated. The isolated GRB2 DNA was ligated into the EcoRI and SacII sites of pGFP2C™ plasmid (Perkin Elmer) to yield pGFP2-Grb2. pGFP2-Grb2 contains a nucleic acid (SEQ ID NO:217) which encodes GFP-Grb2 (SEQ IDNO:218).

To create the SHCP46-GFP fusion protein, Shcp46 cDNA was amplified by PCR using Pfu Turbo polymerase (Stratagene, USA) with the following primers: SHCP46.GFP2.S(CAGCGGAGCTCGTCATGGGACCTGGGGTTTCC)(SEQ ID NO:223) and SHCP46.GFP2.AS (CAGCTGGTACCTCACACTTTCCGATCCACGG)(SEQ ID NO:224) The SHCP46.GFP2.AS primer is engineered to eliminate the SHCP46 cDNA stop codon to facilitate the creation of an in frame fusion to the GFP2 protein. The PCR product was subcloned blunt ended into the TOPO™ vector pCR2.1 (Invitrogen, Carlsbad, Calif.), to create pSHCP46-TOPO. pSHCP46-TOPO was digested with SacI and KpnI and the Shcp46 fragment was isolated. The isolated SHCP46 DNA was ligated into the SacI and KpnI sites of pGFP2C3™ plasmid (Perkin Elmer) to yield pGFP2C3-SHCP46. pGFP2C3-SHCP46 includes a nucleic acid (SEQ ID NO:221) which encodes the GFP2-Shcp46 fusion protein. (SEQ ID NO:222).

To create the STAT5A-GFP fusion, Pfu Turbo polymerase (Stratagene) was used to was used to amplify STAT5A cDNA. The PCR product of the STAT5A coding sequence were cloned into pGFP2-N vector (Biosignal Packard, USA). STAT5A was cloned so that the vector would express a carboxy-terminal STAT5A-GFP2 fusion protein. To create the STAT5A-GFP fusion protein, STAT5 cDNA was amplified by PCR using Pfu Turbo polymerase (Stratagene, USA) with the following primers: STAT5A.GFP2. S(CAGCGGAGCTCGTCATGGGACCTGGGGTTTCC)(SEQ ID NO:233) and STAT5A.GFP2N.S (CAGCTGGTACCTCACACTTTCCGATCCACGG) (SEQ ID NO:234) The STAT5A.GFP2N.AS primer is engineered to eliminate the STAT5A cDNA stop codon to facilitate the creation of an in frame fusion to the GFP2 protein. The PCR product was subcloned blunt ended into the TOPO™ vector pCR2.1 (Invitrogen, Carlsbad, Calif.), to create pSHCP46-TOPO. pSIICP46-TOPO was digested with BgIII and HindIII and the Stat5A fragment was isolated. The isolated STAT5A DNA was ligated into the BgIII and HindIII sites of pGFP2N3™ plasmid (Perkin Elmer) to yield pGFP2N3-SHCP46. pGFP2N3-STAT5A includes a nucleic acid (SEQ ID NO:231) which encodes the GFP2-STAT5A fusion protein. (SEQ ID NO:232).

To assess the ability of BRET to detect interactions of EGFR with known signaling proteins, HEK293T cells were co-transfected with EGFR-Rluc and one of GFP2-Grb2, GFP2-Shcp46, or STAT5A-GFP2 fusion proteins. Two days prior to transfection, HEK293 cells were plated in 10 ml complete DMEM medium at 2×10⁶ cells/10 cm culture dish (Falcon 3003). Cells were transiently transfected using Superfect® (Qiagen) according to the manufacturer's instructions using 2.5 ug EGFR-RLuc DNA with either 40 ug of GFP-Grb2 or GFP-Shcp46, or STAT5A-GFP DNA, respectively. One day after transfection media was changed. Two days after transfection cells were washed once with D-PBS (GIBCO, 14287-080) and 3 ml PBS (with 5 mM EDTA) added. Cells were harvested from the dishes with a cell scraper, washed twice with D-PBS and resuspended to 2×10⁶ cells/ml. Costar 3912, non-treated, white polystyrene, 96-well plates were used for the assay.

To detect agonist activity, 1×10⁶ cells in a 50 μl volume were added per well to the 96-well plate. Various concentrations of EGF (Peprotech, USA) were added to the wells in a 50 μl volume. Cells were incubated for 20 minutes. To detect antagonist activity, 1×10⁶ cells in a 50 μl volume were added per well to the 96-well plate. 25 μl of agonist (EGF), in a final concentration of 2 nM was added to each well. Cells were incubated for 10 minutes. Various concentrations of the EGFR antagonist Iressa were added to the wells in a 25 ∞l volume. Cells were incubated for 10 minutes.

50 μl coelenteracine 400A (Biotium) (5 μM final concentration) was injected into each well to activate the luciferase and initiate the BRET reading. Detection of luminescence emissions from luciferase and GFP2 were performed on a Mithras LB940 plate reader (Berthold, Germany). BRET optimized filters (Berthold, Germany) were used to detect luciferase emission at 395 nm and GFP2 emission at 510 nm wavelength. The luciferase and GFP2 emissions were measured for one second each. Dose response data from the BRT BRET assays were fit to the equation R=D+(A−D)/(1+(x/c)), where A+minimum response, D=maximum response and c=EC50 (R=response, x=concentration of ligand) using the Prism 4.0 software (GraphPad Software, Dan Diego, Calif., USA). All data points represent the mean of three wells.

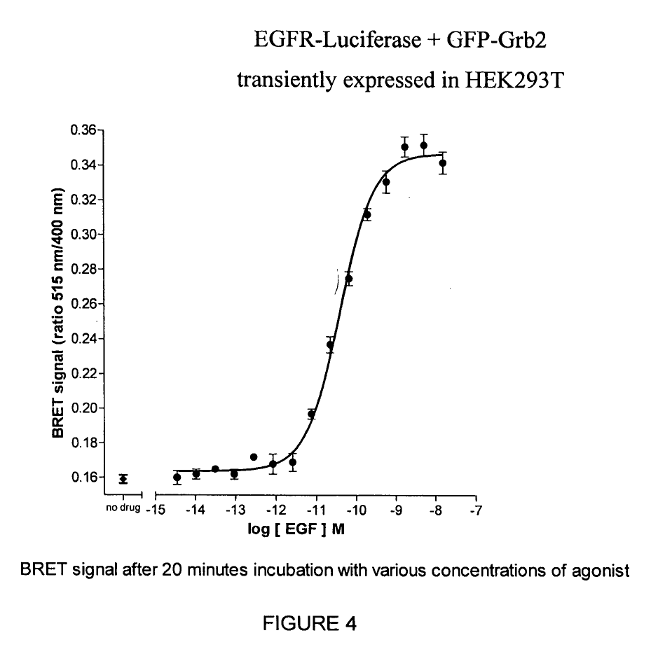

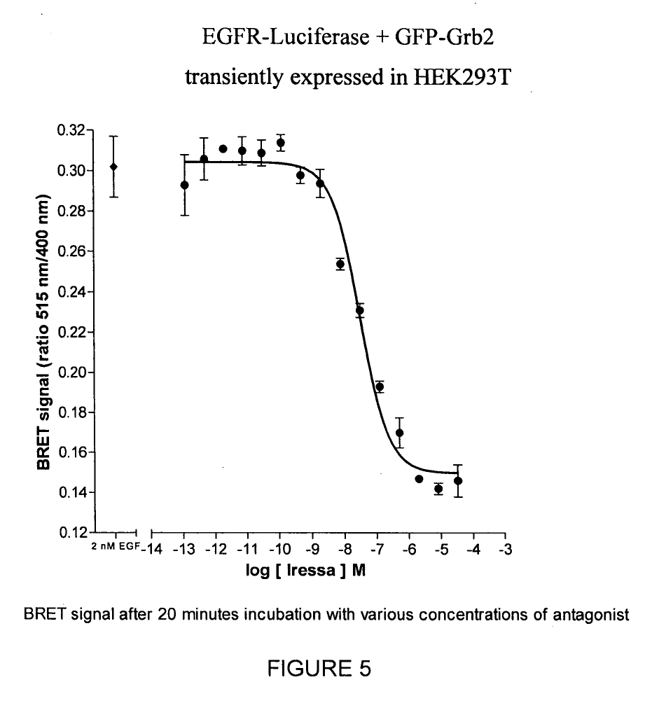

Dose responses are obtained for the EGFR agonist EGF and the EGFR antagonist Iressa (gefitinib) in activating or inhibiting EGFR activity. (FIGS. 4 and 5).

### Example 3

### Applicability of BRET to Identify Ligands That Activate Specific Signaling Pathways

This example demonstrates the utility of BRET technology to detect agonist and antagonist activity on specific RTK signaling pathways. GFP2-Shcp46 and STAT5A-GFP expression vectors are described in Example 1. HEK293T cells were cultured as described in Example 1. Cells were co-transfected with 2.5 μg of EGFR-Rluc and 40 μg of GFP2-GShcp46 or STAT5A-GFP2 vector DNA as described in Example 1. Agonist and antagonist activity is assessed as described in Example 1.

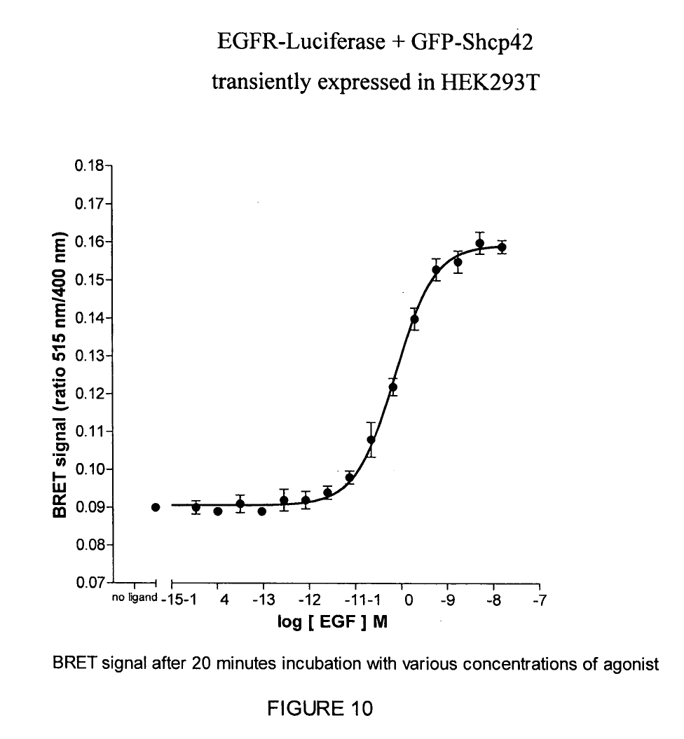

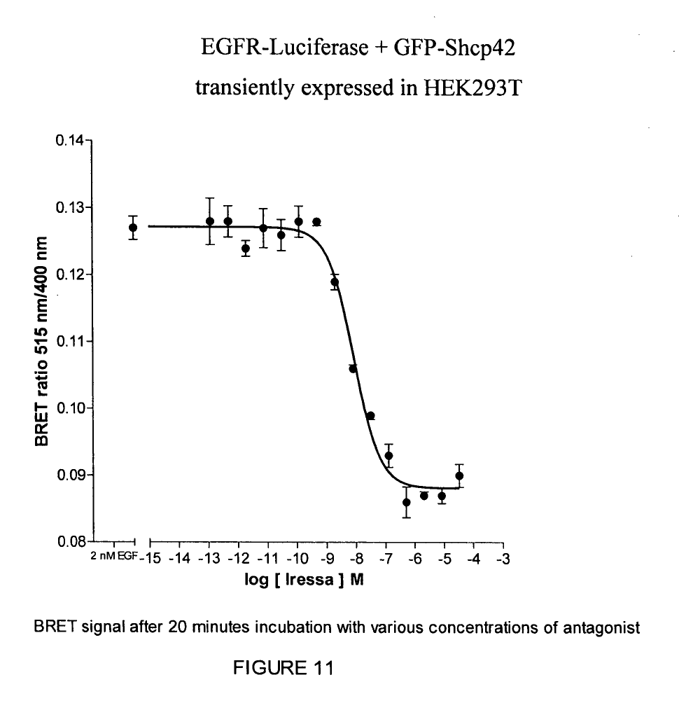

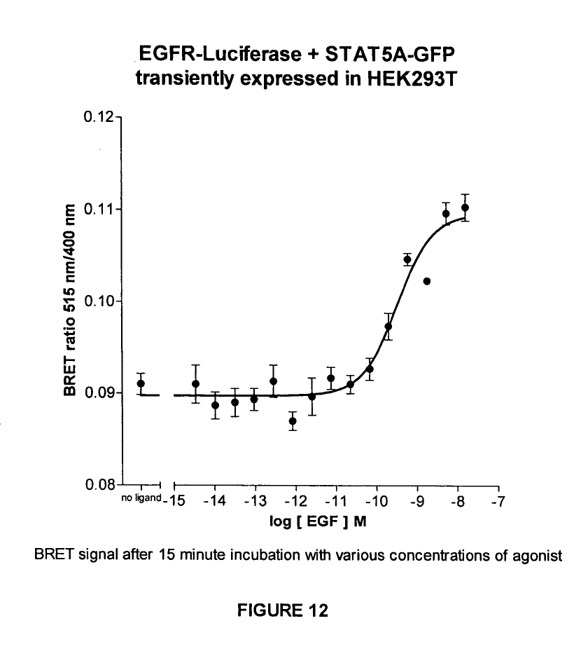

A dose-dependent profile as measured by BRET readings when cells co-expressing EGFR-Rluc and either GFP2-Shcp46 or STAT5A-GFP2, are contacted with varying concentrations of EGFR agonist. (FIG. 10, FIG. 12). FIG. 11 shows a dose-dependent profile as measured by BRET readings when cells co-expressing EGFR-Rluc and GFP2-Shcp46 are treated with varying concentrations of the EGFR antagonist Iressa (gefitinib). The data in FIGS. 10-12 illustrate that a wide variety of RTK signaling proteins can be used in the assay described above. These data also demonstrate that the RTK BRET assay can be utilized to detect signal pathway specific RTK activity.

### Example 4

### Stability of BRET Signal

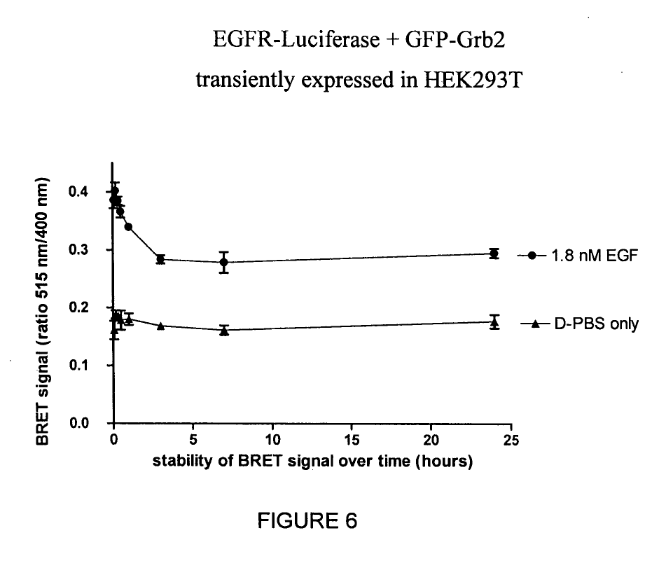

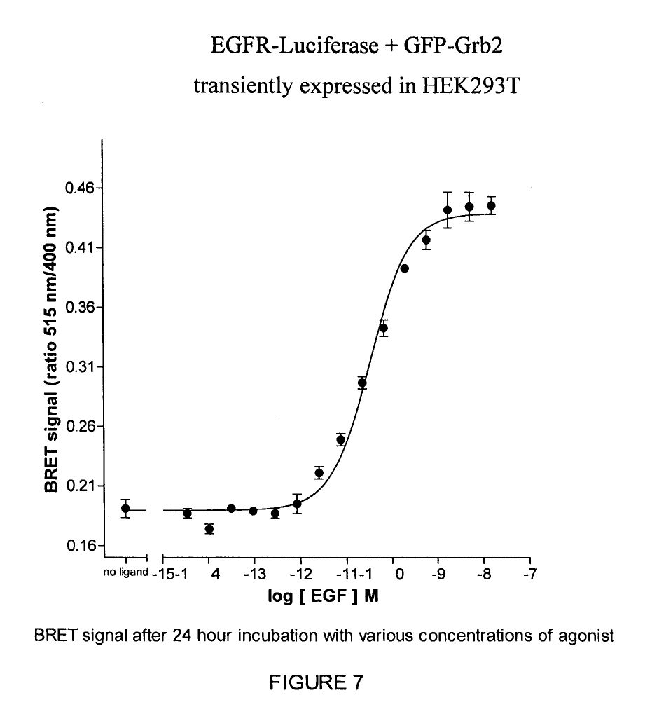

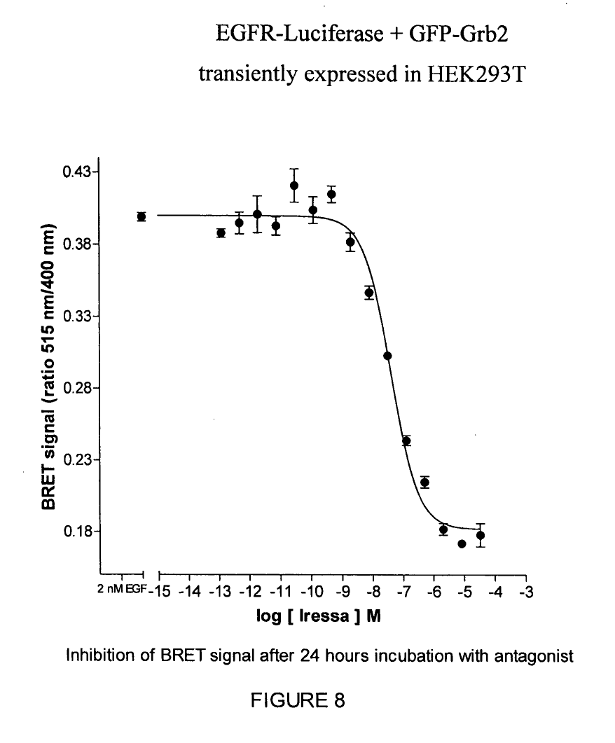

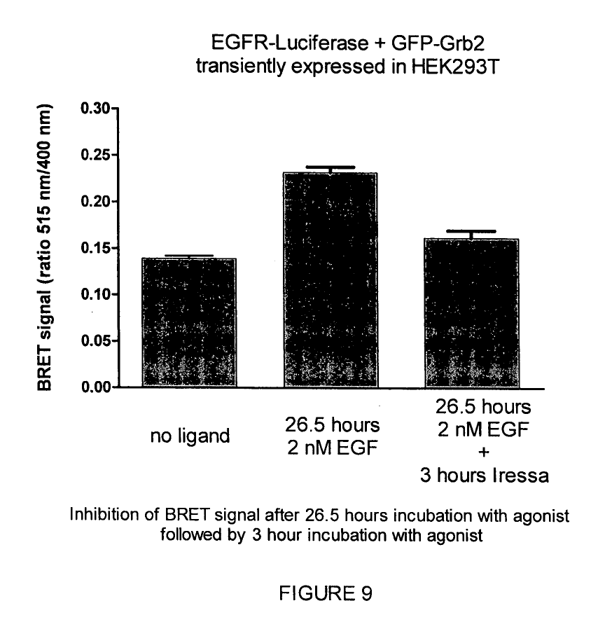

To test the stability of the signal generated in the RTK BRET assay described herein, HEK293 were cultured and co-transfected with EGFR-Rluc and GFP2-Grb2 expression vectors as described in Example 1. Agonist and antagonist assays were performed as described in Example 1. Following initiation of the reaction by the addition of substrate, luminescence emissions from luciferase and GFP2 were measured as described in Example 1 at various timepoints (FIG. 6), after 24 hours (FIG. 7, FIG. 8), or after 26.5 hours (FIG. 9). EGFR mediated BRET signal is stable for more than 24 hours and is still inhibited by the EGFR antagonist Iressa (geniftib) (FIGS. 6, 7, 8 and 9). After 24 hours, EGF and EGF+Iressa (geniftib) continue to show dose-dependent affects on BRET signal. (FIG. 7, FIG. 8). The stability of the BRET signal, as well as the retention of the ability to detect dose-dependent signal renders the BRET assay attractive for screening large compound libraries to identify potential ligands for RTKs, which typically involve long incubation times to prepare mixes of transfected cells and large numbers of test compounds in screening plates.

The disclosures of each of the following references are herein incorporated by reference in their entirety.

### REFERENCES

Akin C, Metcalfe D D. 2004. The biology of Kit in disease and the application of pharmacogenetics. J Allergy Clin Immunol 114(1):13-9; quiz 20.
Bar-Sagi D, Hall A. 2000. Ras and Rho GTPases: a family reunion. Cell 103(2):227-38.
Bellamy W T. 1992. Prediction of response to drug therapy of cancer. A review of in vitro assays. Drugs 44(5):690-708.
Bertrand L, Parent S, Caron M, Legault M, Joly E, Angers S, Bouvier M, Brown M, Houle B, Menard L. 2002. The BRET2/arrestin assay in stable recombinant cells: a platform to screen for compounds that interact with G protein-coupled receptors (GPCRS). J Recept Signal Transduct Res 22(1-4):533-41.
Blume-Jensen P, Hunter T. 2001. Oncogenic kinase signalling. Nature 411(6835):355-65.
Corfas G, Roy K, Buxbaum J D. 2004. Neuregulin 1-erbB signaling and the molecular/cellular basis of schizophrenia. Nat Neurosci 7(6):575-80.
Cunningham M E, Greene L A. 1998. A function-structure model for NGF-activated TRK. Embo J 17(24):7282-93.
Darnell J E, Jr., Kerr I M, Stark G R. 1994. Jak-STAT pathways and transcriptional activation in response to IFNs and other extracellular signaling proteins. Science 264(5164):1415-21.
Downward J. 2004. PI 3-kinase, Akt and cell survival. Semin Cell Dev Biol 15(2):177-82.
Dupriez V J, Maes K, Le Poul E, Burgeon E, Detheux M. 2002. Aequorin-based functional assays for G-protein-coupled receptors, ion channels, and tyrosine kinase receptors. Receptors Channels 8(5-6):319-30.
Farmer G. 2004. Targeted lung cancer therapies. Nat Rev Drug Discov 3(7):547-8.
Garrington T P, Johnson G L. 1999. Organization and regulation of mitogen-activated protein kinase signaling pathways. Curr Opin Cell Biol 11(2):211-8.
Gross M E, Shazer R L, Agus D B. 2004. Targeting the HER-kinase axis in cancer. Semin Oncol 31(1 Suppl 3):9-20.
Haluska P, Adjei A A. 2001. Receptor tyrosine kinase inhibitors. Curr Opin Investig Drugs 2(2):280-6.
Hart R C, Matthews J C, Hori K, Cormier M J. 1979. Renilla reniformis bioluminescence: luciferase-catalyzed production of nonradiating excited states from luciferin analogues and elucidation of the excited state species involved in energy transfer to Renilla green fluorescent protein. Biochemistry 18(11):2204-10.
Heding A. 2004. Use of the BRET 7TM receptor/beta-arrestin assay in drug discovery and screening. Expert Rev Mol Diagn 4(3):403-11.
Heldin C H. 1995. Dimerization of cell surface receptors in signal transduction. Cell 80(2):213-23.
Holbro T, Hynes N E. 2004. ErbB receptors: directing key signaling networks throughout life. Annu Rev Pharmacol Toxicol 44:195-217.
Hubbard SR, Till J H. 2000. Protein tyrosine kinase structure and function. Annu Rev Biochem 69:373-98.
Hunter T. 1998. The role of tyrosine phosphorylation in cell growth and disease. Harvey Lect 94:81-119.
Hunter T. 2000. Signaling-2000 and beyond. Cell 100(1):113-27.
Karin M, Hunter T. 1995. Transcriptional control by protein phosphorylation: signal transmission from the cell surface to the nucleus. Curr Biol 5(7):747-57.
Kenakin T. 1999. The measurement of efficacy in the drug discovery agonist selection process. J Pharmacol Toxicol Methods 42(4):177-87.
Lin N U, Winer E P. 2004. New targets for therapy in breast cancer: small molecule tyrosine kinase inhibitors. Breast Cancer Res 6(5):204-10.
Lorenz W W, McCann R O, Longiaru M, Cormier M J. 1991. Isolation and expression of a cDNA encoding Renilla reniformis luciferase. Proc Nati Acad Sci USA 88(10):4438-42.
Mass R D. 2004. The HER receptor family: a rich target for therapeutic development. Int J Radiat Oncol Biol Phys 58(3):932-40.
McDonald N Q, Murray-Rust J, Blundell T L. 1995. The first structure of a receptor tyrosine kinase domain: a further step in understanding the molecular basis of insulin action. Structure 3(l):1-6.
Mosmann T. 1983. Rapid calorimetric assay for cellular growth and survival: application to proliferation and cytotoxicity assays. J Immunol Methods 65(1-2):55-63.
Muller-Tidow C, Schwable J, Steffen B, Tidow N, Brandt B, Becker K, Schulze-Bahr E, Halfter H, Vogt U, Metzger R and others. 2004. High-throughput analysis of genome-wide receptor tyrosine kinase expression in human cancers identifies potential novel drug targets. Clin Cancer Res 10(4):1241-9.
Olive D M. 2004. Quantitative methods for the analysis of protein phosphorylation in drug development. Expert Rev Proteomics 1 (3):327-341.
Pawson T. 1995. Protein modules and signalling networks. Nature 373(6515):573-80.
Pawson T. 2002. Regulation and targets of receptor tyrosine kinases. Eur J Cancer 38 Suppl 5:S3-10.
Pawson T, Olivier P, Rozakis-Adcock M, McGlade J, Henkemeyer M. 1993. Proteins with SH2 and SH3 domains couple receptor tyrosine kinases to intracellular signalling pathways. Philos Trans R Soc Lond B Biol Sci 340(1293):279-85.
Pawson T, Schlessingert J. 1993. SH2 and SH3 domains. Curr Biol 3(7):434-42.
Pelicci G, Lanfrancone L, Grignani F, McGlade J, Cavallo F, Forni G, Nicoletti I, Pawson T, Pelicci P G. 1992. A novel transforming protein (SHC) with an SH2 domain is implicated in mitogenic signal transduction. Cell 70(1):93-104.
Prenzel N, Zwick E, Leserer M, Ullrich A. 2000. Tyrosine kinase signalling in breast cancer. Epidermal growth factor receptor: convergence point for signal integration and diversification. Breast Cancer Res 2(3): 184-90.
Robinson D R, Wu Y M, Lin S F. 2000. The protein tyrosine kinase family of the human genome. Oncogene 19(49):5548-57.
Sawyer T K, Bohacek R S, Metcalf C A, 3rd, Shakespeare W C, Wang Y, Sundaramoorthi R, Keenan T, Narula S, Weigele M, Dalgarno D C. 2003. Novel protein kinase inhibitors: SMART drug design technology. Biotechniques Suppl:2-10, 12-5.
Schlessinger J. 2000. Cell signaling by receptor tyrosine kinases. Cell 103(2):211-25.
Shawver L K, Slamon D, Ulirich A. 2002. Smart drugs: tyrosine kinase inhibitors in cancer therapy. Cancer Cell 1(2):117-23.
Silva C M. 2004. Role of STATs as downstream signal transducers in Src family kinase-mediated tumorigenesis. Oncogene 23(48):8017-23.
Tsien R Y. 1998. The green fluorescent protein. Annu Rev Biochem 67:509-44.
Ullrich A, Schlessinger J. 1990. Signal transduction by receptors with tyrosine kinase activity. Cell 61(2):203-12.
Verheul H M, Pinedo H M. 2003. Vascular endothelial growth factor and its inhibitors. Drugs Today (Barc) 39 Suppl C:81-93.
Vrecl M, Jorgensen R, Pogacnik A, Heding A. 2004. Development of a BRET2 screening assay using beta-arrestin 2 mutants. J Biomol Screen 9(4):322-33.
Ward W W, Cormier M J. 1979. An energy transfer protein in coelenterate bioluminescence. Characterization of the Renilla green-fluorescent protein. J Biol Chem 254(3):781-8.
Xu Y, Piston D W, Johnson C H. 1999. A bioluminescence resonance energy transfer (BRET) system: application to interacting circadian clock proteins. Proc Natl Acad Sci USA 96(1):151-6.

What is claimed is:

1. A method of evaluating whether a test compound functions as a ligand for a receptor tyrosine kinase, the method comprising:
(i) providing a cell comprising a receptor tyrosine kinase and a second protein, wherein said receptor tyrosine kinase comprises a bioluminescent donor moiety and said second comprises a fluorescent acceptor moiety, and wherein said second protein is found within close physical distance to said receptor tyrosine kinase if said test compound is a ligand for said receptor tyrosine kinase;
(ii) contacting said cell with a test compound; and
(iii) determining whether said receptor tyrosine kinase and said second protein interact in the presence of said test compound.

2. A method of evaluating whether a test compound functions as a ligand for a receptor tyrosine kinase, said method comprising:
(i) providing an cell comprising a receptor tyrosine kinase and a second protein, wherein said receptor tyrosine kinase comprises a bioluminescent donor moiety and said second protein comprises a fluorescent acceptor moiety, wherein said receptor tyrosine kinase and second protein are within close physical distance to each other;
(ii) contacting said cell with a test compound, wherein if said test compound is a ligand for said receptor tyrosine kinase, said receptor tyrosine kinase and said second protein will dissociate such that they are no longer within close physical distance to each other; and
(iii) detecting the interaction between said receptor tyrosine kinase and said second protein.

3. The method of claim 1 or 2, wherein the bioluminescent donor moiety is a luciferase.

4. The method of claim 3, wherein the luciferase is Renilla luciferase.

5. The method of claim 1 or claim 2, wherein the fluorescent acceptor moiety is a GFP moiety.

6. The method of claim 5, wherein the GFP moiety is GFP2.

7. The method of claim 1 or claim 2, wherein the fluorescent acceptor moiety is a YFP moiety.

8. The method of claim 1 or claim 2, wherein the fluorescent acceptor moiety is a CFP moiety.

9. The method of claim 1, wherein the determination step comprises calculating the ratio of light emissions from the fluorescent acceptor moiety and the bioluminescent donor moiety.

10. The method of claim 1, wherein the second protein is a signaling protein that mediates receptor tyrosine kinase signal transduction.

11. The method of claim 2, wherein the second protein is a protein that mediates receptor tyrosine kinase function.

12. The method of claim 10, wherein said signaling protein comprises an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52.

13. The method of claim 10, wherein said signaling protein comprises an amino acid sequence having at least 70% amino acid identity to an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52, wherein said signaling protein retains the ability to interact with a cognate receptor tyrosine kinase.

14. The method of claim 10, wherein said signaling protein comprises at least 5 consecutive amino acids of an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52, wherein said signaling protein retains the ability to interact with a cognate receptor tyrosine kinase.

15. The method of claim 1 or claim 2, wherein said receptor tyrosine kinase comprises an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226.

16. The method of claim 1 or claim 2, wherein said receptor tyrosine kinase comprises an amino acid sequence having at least 70% identity to an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, and 226, and wherein said receptor tyrosine kinase retains the ability to mediate ligand binding and signal transduction.

17. The method of claim 1 or claim 2, wherein said receptor tyrosine kinase comprises at least 5 consecutive amino acids of an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226, and wherein said receptor tyrosine kinase retains the ability to mediate ligand binding and signal transduction.

18. The method of claim 1 or claim 2, wherein the method utilizes bioluminescence resonance energy transfer (BRET) technology.

19. A method of evaluating whether a test compound functions as a ligand for a receptor tyrosine kinase, the method comprising:
(i) providing a cell comprising a receptor tyrosine kinase and a second protein, wherein said receptor tyrosine kinase comprises a fluorescent donor moiety and said second comprises a fluorescent acceptor moiety, and wherein said second protein is found within close physical distance to said receptor tyrosine kinase if said test compound is a ligand for said receptor tyrosine kinase;
(ii) contacting said cell with a test compound; and
(iii) determining whether said receptor tyrosine kinase and said second protein interact in the presence of said test compound

20. A method of evaluating whether a test compound functions as a ligand for a receptor tyrosine kinase, said method comprising:
(i) providing an cell comprising a receptor tyrosine kinase and a second protein, wherein said receptor tyrosine kinase comprises a fluorescent donor moiety and said second protein comprises a fluorescent acceptor moiety, wherein said receptor tyrosine kinase and second protein are within close physical distance to each other;
(ii) contacting said cell with a test compound, wherein if said test compound is a ligand for said receptor tyrosine kinase, said receptor tyrosine kinase and said second protein will dissociate such that they are no longer within close physical distance to each other; and
(iii) detecting the interaction between said receptor tyrosine kinase and said second protein.

21. The method of claim 19 or 20, wherein said receptor tyrosine kinase is a fusion protein comprising a tyrosine kinase fused to said fluorescent donor moiety.

22. The method of claim 19, wherein the determination step comprises calculating the ratio of light emissions from the fluorescent acceptor moiety and the fluorescent donor moiety.

23. The method of claim 19 or 20, wherein the second protein is a signaling protein that mediates receptor tyrosine kinase signal transduction.

24. The method of claim 23, wherein said signaling protein comprises an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52.

25. The method of claim 23, wherein said signaling protein comprises an amino acid sequence having at least 70% amino acid identity to an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52, wherein said signaling protein retains the ability to interact with a cognate receptor tyrosine kinase.

26. The method of claim 23, wherein said signaling protein comprises at least 5 consecutive amino acids of an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52, wherein said signaling protein retains the ability to interact with a cognate receptor tyrosine kinase.

27. The method of claim 19 or 20, wherein said receptor tyrosine kinase comprises an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226.

28. The method of claim 19 or 20, wherein said receptor tyrosine kinase comprises an amino acid sequence having at least 70% identity to an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, and 226, and wherein said receptor tyrosine kinase retains the ability to mediate ligand binding and signal transduction.

29. The method of claim 19 or 20, wherein said receptor tyrosine kinase comprises at least 5 consecutive amino acids of an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166. 168, 170, 172, 174, 176, 178, 180, 182, 184, 186, 188, 190, 192, 194, 196, 198, 200, 202, 204, 206, 208, 210, 212, 214, 21 6, 218, 220, 222, 224, or 226, and wherein said receptor tyrosine kinase retains the ability to mediate ligand binding and signal transduction.

30. The method of claim 19 or 20, wherein the method utilizes fluorescent resonance energy transfer (FRET) technology.

31. A method of assessing the effect of a test compound on the activity of a receptor tyrosine kinase comprising:
(iv) providing a cell expressing one or more receptor tyrosine kinases and one or more second proteins, wherein said receptor tyrosine kinase comprises a bioluminescent donor moiety and said second protein comprises a fluorescent acceptor moiety, and wherein at least one of said receptor tyrosille kinases or second proteins is expressed from a nucleic acid which has been introduced into said cell;
(v) contacting said cell with said test compound; and
(vi) determining whether said test compound influences the activity of said receptor tyrosine kinase.

32. The method of claim 31 wherein said determining step comprises determining whether said test compound is an agonist.

33. The method of claim 31 wherein said determining step comprises determining whether said test compound is an antagonist.

34. The method of claim 31 wherein said determining step comprises determining whether said test compound is an inverse agonist.

35. The method of claim 31 wherein said determining step comprises determining whether said test compound is a selective modulator.

36. The method of claim 31 wherein the test compound is a naturally occurring compound.

37. The method of claim 31, wherein the test compound is a synthetic compound.

38. An isolated cell comprising a receptor tyrosine kinase and a second protein, wherein said receptor tyrosine kinase comprises a bioluminescent donor moiety fusion protein and wherein said second protein comprises a fluorescent acceptor moiety fusion protein, and wherein modulation of the activity of said receptor tyrosine kinase affects the protein protein interactions between said receptor tyrosine kinase and said second protein.

39. An isolated cell comprising a receptor tyrosine kinase and a second protein, wherein said receptor tyrosine kinase comprises a fluorescent donor moiety fusion protein and wherein said second protein comprises a fluorescent acceptor moiety fusion protein, wherein said fluorescent donor moiety and said fluorescent acceptor moiety are different, and wherein modulation of the activity of said receptor tyrosine kinase affects the protein protein interactions between said receptor tyrosine kinase and said second protein.

40. The cell of claim 38, wherein said receptor tyrosine kinase is a fusion protein comprising a receptor tyrosine kinase fused to a bioluminescent protein.

41. The cell of claim 40, wherein said wherein said bioluminescent donor moiety is a luciferase.

42. The cell of claim 41, wherein said luciferase is Renilla luciferase.

43. The cell of claim 38 or 39, wherein said fluorescent acceptor moiety is a GFP moiety.

44. The cell of claim 43, wherein said GFP moiety is GFP2.

45. The cell of claim 38 or claim 39, wherein said fluorescent acceptor moiety is a YFP moiety.

46. The cell of claim 38 or claim 39, wherein said fluorescent acceptor moiety is a CFP moiety.

47. The cell of claim 38 or claim 39, wherein said second protein is a signaling protein that mediates receptor tyrosine kinase signal transduction.

48. The cell of claim 47, wherein said signaling protein comprises an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52.

49. The cell of claim 47, wherein said signaling protein comprises an amino acid sequence having at least 70% amino acid identity to an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52, wherein said signaling protein retains the ability to interact with a cognate receptor tyrosine kinase.

50. The cell of claim 47, wherein said signaling protein comprises at least 5 consecutive amino acids of an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, and 52, wherein said signaling protein retains the ability to interact with a cognate receptor tyrosine kinase.

51. The cell of claim 38 or claim 39, wherein said receptor tyrosine kinase comprises an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226.

52. The cell of claim 38 or claim 39, wherein said receptor tyrosine kinase comprises an amino acid sequence having at least 70% identity to an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226, and wherein said receptor tyrosine kinase retains the ability to mediate ligand binding and signal transduction.

53. The cell of claim 38 or claim 39, wherein said receptor tyrosine kinase comprises at least 5 consecutive amino acids of an amino acid sequence selected from the group consisting of SEQ ID NOs: 110, 112, 114, 116, 118, 120, 122, 124, 126, 128, 130, 132, 134, 136, 138, 140, 142, 144, 146, 148, 150, 152, 154, 156, 158, 160, 162, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, or 226, and wherein said receptor tyrosine kinase retains the ability to mediate ligand binding and signal transduction.

54. The cell of claim 38 or claim 39, wherein said isolated cell is a eukaryotic cell.

55. The cell of claim 38 or claim 39, wherein said isolated cell is a mammalian cell.

56. A method of evaluating whether a test compound functions as a ligand for a receptor tyrosine kinase, said method comprising:
(i) providing an isolated cell comprising a receptor tyrosine kinase fused to a bioluminescent donor moiety and a second protein fused to a fluorescent acceptor moiety,
(ii) contacting the cell with a substrate, wherein the bioluminescent donor moiety on said receptor tyrosine kinase emits light at a first wavelength in the presence of said substrate, wherein the energy emitted from the bioluminescent donor moiety is transferred to said fluorescent acceptor moiety on said second protein when said fluorescent acceptor moiety is in close proximity to said bioluminescent donor moiety, and wherein said fluorescent acceptor moiety emits light at a second wavelength when said bioluminescent donor moiety transfers energy to said fluorescent acceptor moiety;
(iii) contacting said cell with a test compound; and
(iv) measuring the emission of light at said first wavelength and at said second wavelength; and

57. A method of making a pharmaceutical composition comprising
(i) performing the method of any one of claims 1 or 19;
(ii) identifying a test compound that is an agonist of said receptor tyrosine kinase; and
(iii) combining said test compound with a pharmaceutically acceptable carrier.

58. A method of making a pharmaceutical composition comprising
(i) performing the method of any one of claims 1 or 19;
(ii) identifying a test compound that is an antagonist of said receptor tyrosine kinase; and
(iii) combining said test compound with a pharmaceutically acceptable carrier.

59. A method of making a pharmaceutical comprising
(i) performing the method of any one of claims 1 or 19;
(ii) identifying a test compound that is an inverse agonist of said receptor tyrosine kinase; and
(iii) combining said test compound with a pharmaceutically acceptable carrier.

60. A method of making a pharmaceutical comprising
(i) performing the method of any one of claims 1 or 19;
(ii) identifying a test compound that is an selective modulator of said receptor tyrosine kinase; and
(iii) combining said test compound with a pharmaceutically acceptable carrier

61. A pharmaceutical composition comprising a compound identified by any one of claims 1 or 19, and a physiologically acceptable carrier, diluent, or excipient, or a combination thereof.

62. The method of claim 10, wherein said signaling protein comprises an amino acid selected from the group consisting of SEQ ID NOs: 2, 26, and 230.

63. The method of claim 1 or 2, wherein said receptor tyrosine kinase comprises the amino acid sequence of SEQ ID NO: 110.

64. The cell of claim 47, wherein said signaling protein comprises an amino acid selected from the group consisting of SEQ ID NOs: 2, 26, and 230.

65. The cell of claim 38 or 39, wherein said receptor tyrosine kinase comprises the amino acid sequence of SEQ ID NO: 110.
